(19) United States
(12) Patent Application Publication
(10) Pub. No.: US 2009/0032423 A1
(43) Pub. Date: Feb. 5, 2009

(54) ANTIMICROBIAL MEDICAL WASTE DISPOSAL ENCLOSURES

(76) Inventor: John Japuntich, Harvard, IL (US)

Correspondence Address: RATNERPRESTIA, P.O. BOX 980, VALLEY FORGE, PA, 19482, US

(21) Appl. No.: 12/088,044

(22) PCT Filed: Sep. 25, 2006
(86) PCT No.: PCT/US2006/037190

§ 371 Date: Aug. 26, 2008

Related U.S. Application Data
(60) Provisional application No. 60/720,557, filed on Sep. 26, 2005.

(51) Int. Cl. B65D 83/00; A61B 17/06; B65D 81/00

(52) U.S. Cl. 206/366; 206/438; 220/23.83

(57) ABSTRACT

An enclosure configured to receive and secure a medical waste container. The enclosure comprises a main body and a cover. The cover is connected to the main body and configured to be moved between an opened position and a closed position. An antimicrobial agent is provided along the inside and outside surfaces of the main body and along the inside and outside surfaces of the cover to inhibit growth of bacteria and the like.

### BACKGROUND OF THE INVENTION

The present invention relates to medical waste disposal systems. More particularly, the present invention relates to enclosures or other hardware configured to be used with medical waste containers.

In hospitals, clinics and similar institutions, contamination is of utmost concern. Disposable, single use patient-care products, which used to be the exception, are now the norm. Current guidelines of the U.S. Center for Disease Control require immediate disposal of hypodermic needles or other sharps at their site of use immediately following an injection.

Many different types of containers are available for sharps disposal, including many manufactured by Tyco Healthcare, Inc. In many environments, it is desirable to secure the container to protect against an individual gaining access to sharps or other materials within the container.

To secure the disposable containers, various enclosure systems have been developed. Many enclosure systems include a hollow, outer enclosure configured to receive and secure the disposable container located within the outer enclosure. An elongated slot or the like is provided at the top of the enclosure for permitting access to its interior. The disposable container includes an inlet in registration with the slot to allow disposal of contaminated items therein. The outer enclosure is typically provided with an access door in order to permit easy removal and insertion of the disposable container into the outer enclosure. While the door is often provided in the front side of the enclosure, other configurations, such as hinging the top of the enclosure, are also utilized. Furthermore, the enclosures can have various configurations to accommodate various disposable containers. For example, the enclosures may have integrally formed hoods or the like. Additionally, secondary enclosures, for example, a secondary enclosure to house gloves or the like, may be attached to the primary enclosure. Illustrative enclosures are shown in U.S. Pat. Nos. 4,715,498; 5,211,303; Des. 385,349; and Des. 385,626, each of which is incorporated herein by reference.

In may areas of a hospital or the like, high volumes of sharps are necessary in delivering patient care, which results in high volumes of waste. Given the increased waste, larger disposable containers are utilized. To accommodate and secure the larger containers, enclosures in the form of moveable carts have been developed. Most recently, such carts have included a molded frame configured to support and secure the containers. Illustrative cart enclosures are shown in U.S. Pat. No. Des. D478,701 and U.S. Published Patent Application No. US2002/0158068A1, each of which is incorporated herein by reference.

Other hardware is used in areas of a hospital or the like in connection with the disposal of medical sharps. For example, enclosures are often provided to house disposable goods such as the gloves worn by medical professionals and staff when handling medical waste. Such glove enclosures (or glove boxes) are often associated with or are components of medical waste disposal systems.

While secured disposal containers and associated equipment (e.g., carts and glove boxes and other hardware associated with medical waste containers) have generally reduced contamination and misuse of sharps and other medical waste, additional improvements are sought in this field.

### SUMMARY OF THE INVENTION

The present invention provides an enclosure configured to receive and secure a medical waste container. The enclosure comprises a main body having a body is inside surface and a body outside surface and a cover having a cover inside surface and a cover outside surface. The cover is connected to the main body and configured to be moved between an opened position and a closed position. An antimicrobial agent is provided along both the body inside surface and the body outside surface and along both the cover inside surface and the cover outside surface. The antimicrobial agent inhibits growth of bacteria and the like.

According to another aspect of the invention, a reusable apparatus is provided for use in connection with the disposal of medical waste. The reusable apparatus includes a body at least partially defining an interior region configured to accommodate medical waste or supplies used in conjunction with medical waste. The reusable apparatus also includes an antimicrobial agent associated with the body in an amount effective to resist the growth of microbes on an inner surface of said body facing the interior region and an outer surface of the body facing away from the interior region.

The reusable apparatus can be, for example, a wall enclosure for a sharps disposal container, a glove box associated with a sharps disposal container, a cart for a sharps disposal container, or a reusable medical waste disposal container. The reusable apparatus is optionally formed from plastic, and the antimicrobial agent is optionally blended in with the plastic. Also, the antimicrobial agent is optionally associated with the body in an amount effective to resist odor emanating from the body.

According to another aspect of the invention, a medical waste disposal system includes a disposable medical waste container configured to receive medical waste for disposal and a reusable enclosure housing the disposable medical waste container. The reusable enclosure has a body at least partially defining an interior surface facing the disposable medical waste container, a door movable between opened and closed positions, and an antimicrobial agent associated with the body and the door in an amount effective to resist the growth of microbes on the body or the door.

According to yet another aspect of the invention, a medical waste disposal system includes a medical waste container configured to receive medical waste for disposal, a disposable supply of gloves configured for use in connection with the handling of the medical waste, and a reusable glove box mounted with the medical waste container and housing the disposable supply of gloves. The glove box has a body at least partially defining an interior surface facing the disposable supply of gloves and an antimicrobial agent associated with the body in an amount effective to resist the growth of microbes on the body.

According to still another aspect of the invention, a medical waste disposal system includes a disposable medical waste container configured to receive medical waste for disposal and a reusable cart housing the disposable medical waste container. The reusable cart has a body at least partially defining an interior surface facing the disposable medical waste container and an antimicrobial agent associated with the body in an amount effective to resist the growth of microbes on the body.

According to yet another aspect of the invention, a medical waste disposal system includes a reusable medical waste container configured to receive medical waste for disposal, to be emptied of medical waste, and thereafter, to be reused to receive medical waste for disposal. The reusable waste container has a body at least partially defining an interior surface facing the received medical waste material and an antimicrobial agent associated with the body in an amount effective to resist the growth of microbes on the body. The reusable waste container may be a free-standing unit, supported by a bracket or the like, or positioned in an enclosure, the enclosure preferably including an antimicrobial agent as described herein. The reusable waste container is preferably removed from the clinical environment to be emptied and then returned to the clinical environment for reuse.

### BRIEF DESCRIPTION OF THE DRAWINGS

FIG. 1 is a front elevational view, with portions cut away, illustrating a sharps disposal system incorporating an enclosure according to a first exemplary embodiment of the invention.

FIG. 2 is a cross-sectional view taken along lines 2-2 of FIG. 1.

FIG. 3 is a perspective view of an enclosure that is an alternate embodiment of the present invention.

FIG. 4 is a perspective view of an enclosure that is another alternate embodiment of the present invention.

FIG. 5 is a perspective view illustrating a sharps disposal system incorporating an enclosure according to an alternate embodiment of the invention.

FIG. 6 is a perspective view of the enclosure body of the enclosure of FIG. 5.

### DETAILED DESCRIPTION OF THE INVENTION

While various embodiments of the invention are shown and described herein, it will be understood that such embodiments are provided by way of example only. Numerous variations, changes and substitutions will occur to those skilled in the art without departing from the spirit of the invention.

In the most basic form of the present invention, the disposal system includes an enclosure, or other hardware associated with the handling or disposal of medical waste, including a broad spectrum antimicrobial agent associated therewith to inhibit bacterial, fungal, viral and/or other pathogen growth. According to one embodiment, the enclosure includes a main body and a closeable door. Preferably, an antimicrobial agent is associated or incorporated into the polymeric material from which the enclosure is made. Thus, an effective amount of an antimicrobial substance, for example, but not limited to 5-chloro-2-(2,4-dichlorophenoxy)phenol, is incorporated therein.

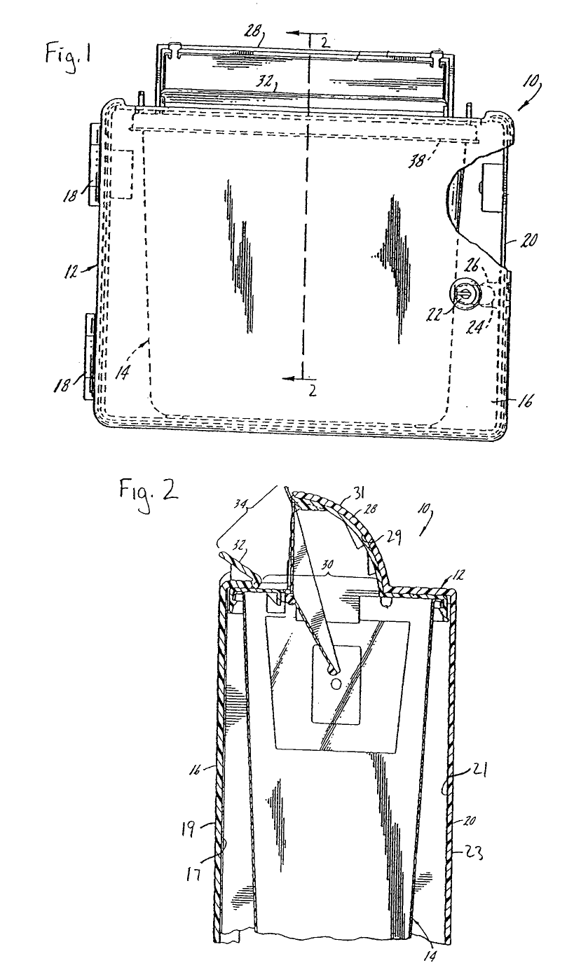

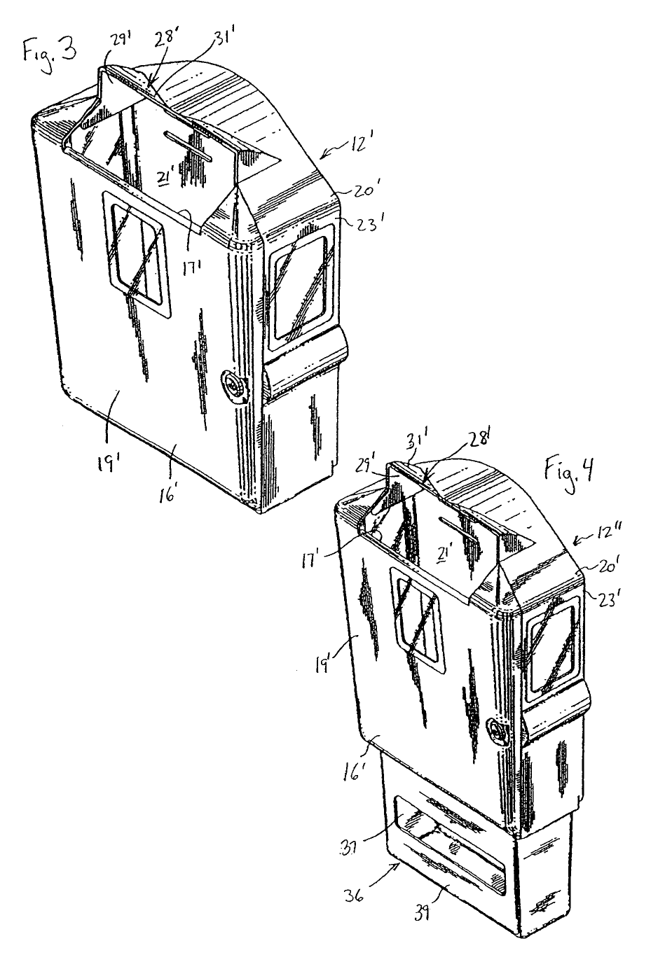

A medical waste disposal system 10 incorporating an enclosure 12 that is a first exemplary embodiment of the invention is shown in FIGS. 1 and 2. The enclosure 12 is configured to receive and secure a disposable container 14. The exemplary enclosure 12 includes an access door 16 secured by hinges 18 onto the main body 20. Opposite the hinges 18, the door 16 includes a lock 22 having a rotatable latch 24 which may be engaged behind a lug 26 extending inwardly from the body 20. When the latch 24 is rotated horizontally (as illustrated in FIG. 1), the latch 24 is engaged behind the lug 26, and the door 16 is locked. When the latch 24 is rotated to a vertical orientation, the lug 26 is no longer engaged, and the door 16 may be opened.

The main body 20 of the outer enclosure 12 includes a raised cowl 28 extending over a slot 30 in the top of the body 20. The cowl 28 constricts access to the slot 30 in order to restrict access to the interior of the outer enclosure 12. The door 16 also includes a raised shelf 32 extending along one side of the slot 30. The combination of the shelf 32 and the cowl 28 comprises a barrier restricting access to the interior of the outer enclosure 12. The relative spacing of the shelf 32 and the cowl 28 defines a limited opening 34 which restricts access to the interior of the enclosure 12. The opening 34 is dimensioned so that, in combination with the inner container 14, access is inhibited to the extent that it is difficult or impossible for an adult human hand to pass through the opening 34. The enclosure 12 can have various configurations, and is not limited to the above described configuration. For example, the enclosure 12 may have a generally open top with the disposable container 14 providing the access limiting mechanism. In the alternate embodiment of the enclosure 12′ illustrated in FIG. 3, the main body 20′ and the door 16′ have raised top surfaces such that the cowl 28′ is formed integrally within the shape of the enclosure 12′. The present invention is not limited to any specific configuration of the enclosure 12.

The door 16, main body 20 and cowl 28 of the enclosure 12 are desirably molded or otherwise formed from a suitable polymer, for example, but not limited to, polypropylene, linear low density polyethylene (LLDPE), styrene, polystyrene, polyethylene, ultra high molecular weight polyethylene, acrylonitrile-butadiene-styrene (ABS), polyester, polyester elastomer, polyester urethane, ethylene/propylene alloy, silicone, polyvinyl chloride, chlorinated polyvinyl chloride, polyvinylidene fluoride, vinyl esters, cellulose acetate proprionate (CAP), polycarbonate, polyolefin alloy, thermoplastic and thermosetting plastic. The material is desirably a stiff material which does not readily deform in use.

To reduce the growth of bacteria or the like on the enclosure 12, an antimicrobial agent is blended into the polymer prior to formation of the enclosure components. The antimicrobial substance may be a chlorinated phenol (e.g., 5-chloro-2-(2,4-dichlorophenoxy)phenol). An alternative antimicrobial agent is polyhexamethylene biguanide hydrochloride (PHMB). The present invention is not limited to any specific antimicrobial agent and other chemical compounds having known antimicrobial characteristics may also be used. As used herein, an antimicrobial agent is an agent that destroys or inhibits the growth of microorganisms. Exemplary antimicrobial agents are available from Microban International, Ltd. of New York, N.Y. The preferred method of associating the antimicrobial agent with the enclosure 12 is to incorporate the antimicrobial agent into a synthetic, polymeric master batch prior to forming the components. Since the antimicrobial agent is mixed into the material prior to forming, the antimicrobial protection is provided along the inside surface 17 and the outside surface 19 of the cover 16, the inside surface 21 and the outside surface 23 of the main body 20, and the inside surface 29 and the outside surface 31 of the cowl 28.

While prior art disposal systems have attempted to reduce contamination by placing a pad or the like having a contaminant neutralizing substance within the disposable container, see U.S. Pat. No. 4,936,449, such products failed to recognize the potential contamination of the environment around the disposable container. This is a particular problem with the enclosures used to secure the disposable containers since the enclosures are generally permanent fixtures which are not disposed of and replaced frequently. Failure to continually clean the enclosure components can lead to the growth of bacteria and the like which can pose health risks, cause undesired odors or reduce the aesthetic appearance of the enclosure. By providing the antimicrobial substance on both the inside surfaces 17, 21, 29 and outside surfaces 19, 23, 31 of the enclosure 12 components, the potential for bacterial growth, and the potential migration from the interior surface to an externally exposed surface, is greatly reduced.

The present invention, according to one exemplary embodiment, provides a reusable apparatus for use in connection with the disposal of medical waste. The reusable apparatus includes a body at least partially defining an interior region configured to accommodate medical waste or supplies used in conjunction with medical waste. The reusable apparatus also includes an antimicrobial agent associated with the body in an amount effective to resist the growth of microbes on an inner surface of said body facing the interior region and an outer surface of the body facing away from the interior region. In this way, the reusable apparatus reduces the potential from microbes generated within the interior region from migrating to the outside surface. Additionally, the exterior surface resists the growth of microbes that might directly contact the outside surface during disposal of medical waste or removal of medical supplies.

According to one aspect, embodiments of this invention are particularly beneficial in the field of medical waste disposal for applications in which a reusable article is used in conjunction with a disposable article. To illustrate, these benefits are pronounced for applications in which a reusable article is used to contain or otherwise house or accommodate or engage a disposable article that can be separated from the reusable article for disposal and replacement. Examples include a reusable enclosure used to house a disposable medical waste container that is removed from the reusable enclosure for disposal and replacement when it is filled with medical waste; a reusable box-like housing used to accommodate a disposable glove supply (such as a glove box) that is taken out of the reusable box-like housing for disposal and replacement when the supply is depleted; a reusable cart used to carry a disposable medical waste container that is removed from the reusable cart for disposal and replacement when it is filled with medical waste; a reusable medical waste container configured to receive medical waste for disposal, to be emptied of medical waste, and thereafter, to be reused to receive medical waste for disposal; or any other reusable article used in combination with a disposable article. Such applications are especially susceptible to microbial growth in the medical environment because of the necessary handling of both the reusable and disposable components of such medical waste systems not only during normal use but also during removal of the disposable component from the reusable component for replacement.

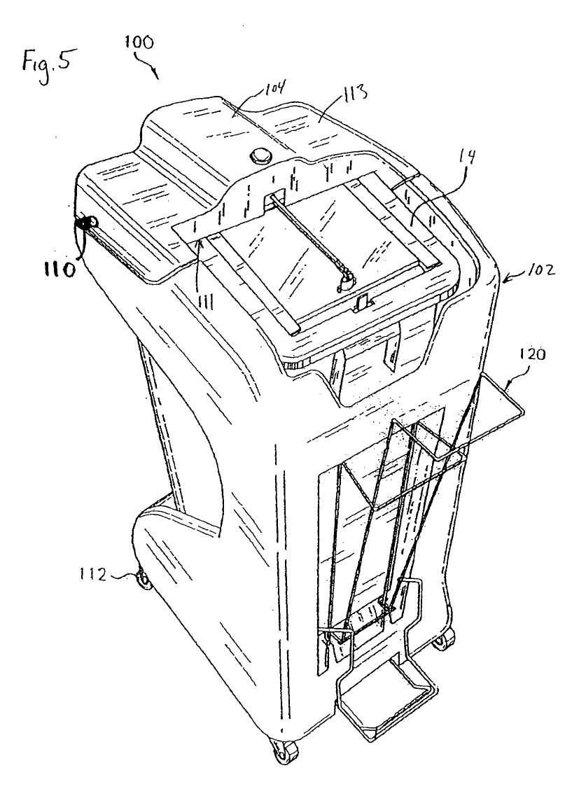

Referring to FIGS. 3 and 4, enclosures 12′ and 12″ that are alternate embodiments of the present invention are shown. As explained above, enclosure 12′ is similar to the enclosure 12 of the previous embodiment, and includes a main body 20′ with a hinged door 16′. In the present embodiment, the main body 20′ and the door 16′ have raised top surfaces such that the cowl 28′ is formed integrally within the shape of the enclosure 12′. The primary enclosure portion of enclosure 12″ is the same as enclosure 12′ and includes the door 16′, the main body 20′ and the cowl 28′. Enclosure 12″ further includes a secondary enclosure body 36 having an inside surface 37 and an outside surface 39. The secondary enclosure body 36 is configured to house a glove box or the like.

As in the previous embodiment, the enclosure components are preferably manufactured from a polymer with an antimicrobial agent blended therein. Thus, the antimicrobial agent is provided along the inside surface 17′ and the outside surface 19′ of the cover 16′, the inside surface 21′ and the outside surface 23′ of the main body 20′, and the inside surface 29′ and the outside surface 31′ of the cowl 28′ of both enclosures 12′ and 12″. Furthermore an antimicrobial agent is provided along the inside surface 37 and the outside surface 39 of the secondary enclosure body 36.

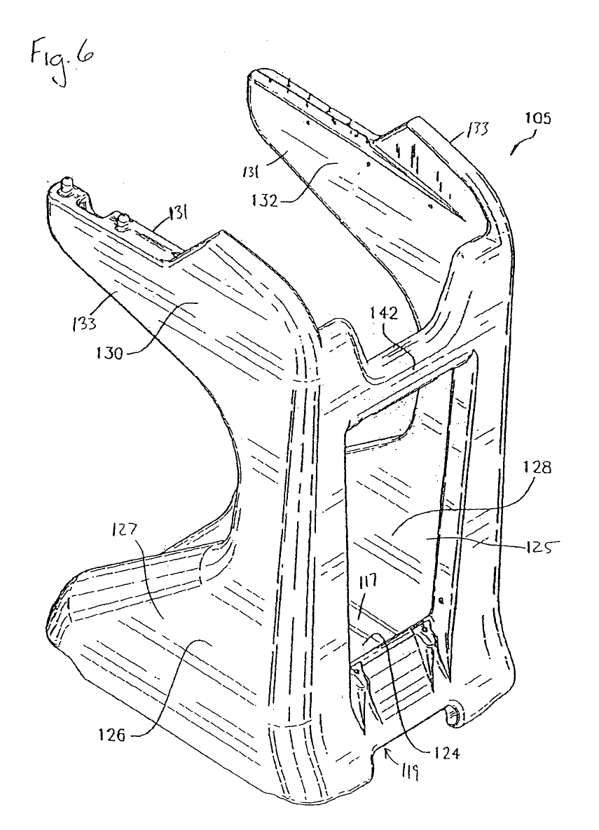

Referring to the FIGS. 5 and 6, a disposal system 100 incorporating an enclosure 102 that is another alternate embodiment of the invention will be described. The enclosure 102 is a cart that is configured to hold a medical waste container 14. The enclosure 102 includes a body 105 that is adapted to receive the medical waste container 14. The illustrated enclosure includes four (4) wheels 112 mounted at the base of the body 105 so that the medical waste container system 100 can easily and readily be transported from one position to another by a user of the system 100. Two (2) of the four (4) wheels 112 are preferably lockable so that movement of the system 10 along the ground can be prevented or controlled. An extendable handle 120 extends upwardly and outwardly from the carrier 100 so that the container system 10 can be pulled by a user. The handle 120 is formed from thick metallic wire formed into an elongated loop. The wire handle 120 may be removed from the body 105 and easily cleaned.

The enclosure also includes a cover 104 that is coupled by means of a hinge (not shown) to the body 105 of the enclosure 102. The hinge makes it possible to rotate the cover 104 with respect to the body 105 so that the cover 104 can be moved between an opened position and a closed position (as shown in FIG. 5). When the cover 104 is rotated into the closed position, removal of the medical waste container 14 from the enclosure 102 is prevented. Also, a lock 110 is preferably provided on the cover 104 in order to lock the cover 104 in a closed position with respect to the body 105 of the enclosure 102. Lock 110 can be a simple key lock, wherein rotation of the key brings about (or releases) engagement between the cover 104 and the body 105.

FIGS. 5 and 6 illustrate one embodiment of the body component 105 of the enclosure 102. The body 105 of the carrier 100 includes a base portion 124 to which the wheels 112 can be mounted. The body 105 also includes a pair of lower side portions 126 and 128, a pair of upper side portions 130 and 132, and an upper front portion 142 that extends between the upper side portions 130 and 132. It should be noted that the body 105 could be formed in a wide variety of shapes, configurations, materials, sizes, and proportions. The shape and configuration of the body 105 are not dictated by its function. Instead, the configuration of the body 105 provides the enclosure 102 with an ornamental appearance that is appealing to the eye.

The body 105 and the cover 104 are preferably formed from plastic such as polypropylene, linear low density polyethylene (LLDPE), styrene, polystyrene, polyethylene, ultra high molecular weight polyethylene, acrylonitrile-butadiene-styrene (ABS), polyester, polyester elastomer, polyester urethane, ethylene/propylene alloy, silicone, polyvinyl chloride, chlorinated polyvinyl chloride, polyvinylidene fluoride, vinyl esters, cellulose acetate proprionate (CAP), polycarbonate, polyolefin alloy, thermoplastic and thermosetting plastic. The material is desirably a stiff material which does not readily deform in use. The body 105 is preferably formed by a molding process, for example, a rotational molding process, however, other manufacturing process may be utilized.

As in the previous embodiments, to reduce the growth of bacteria or the like on the enclosure 102, an antimicrobial agent is blended into the polymer prior to formation of the body 105 and cover 104. Various antimicrobial agents may be utilized. The preferred method of associating the antimicrobial agent with the enclosure 12 is to incorporate the antimicrobial agent into a synthetic, polymeric master batch prior to forming the components. The antimicrobial protection is thereby provided along the inside surface 117 and the outside surface 119 of base portion 124; the inside surface 125 and the outside surface 127 of each lower side portion 126, 128, the inside surface 131 and the outside surface 133 of each of upper side portion 130, 132, the inside surface 143 and the outside surface 145 of the upper front portion 142, and the inside surface 111 and the outside surface 113 of the cover 104.

Although the invention is illustrated and described herein with reference is to specific embodiments, the invention is not intended to be limited to the details shown. Rather, various modifications may be made in the details within the scope and range of equivalents of the claims and without departing from the invention.

What is claimed:

1. A reusable apparatus configured for use in connection with the disposal of medical waste, the reusable apparatus comprising:
a body at least partially defining an interior region configured to accommodate medical waste or supplies used in conjunction with medical waste; and
an antimicrobial agent associated with the body in an amount effective to resist the growth of microbes on an inner surface of said body facing said interior region and an outer surface of said body facing away from said interior region.

2. The reusable apparatus of claim 1, wherein said apparatus is selected from the group consisting of a wall enclosure for a sharps disposal container, a glove box associated with a sharps disposal container, and a cart for a sharps disposal container.

3. The reusable apparatus of claim 1, wherein said body is formed from plastic and said antimicrobial agent is blended in with the plastic.

4. The reusable apparatus of claim 1, wherein said antimicrobial agent associated with the body in an amount effective to resist odor emanating from the body.

5. An enclosure configured to receive and secure a medical waste container, the enclosure comprising:
a main body having a body inside surface and a body outside surface;
a cover having a cover inside surface and a cover outside surface, the cover connected to the main body and configured to be moved between an opened position and a closed position; and
an antimicrobial agent provided along both the body inside surface and the body outside surface and along both the cover inside surface and the cover outside surface.

6. A medical waste disposal system comprising:
a disposable medical waste container configured to receive medical waste for disposal;
a reusable enclosure housing said disposable medical waste container, said reusable enclosure having a body at least partially defining an interior surface facing said disposable medical waste container, a door movable between opened and closed positions, and an antimicrobial agent associated with said body and said door in an amount effective to resist the growth of microbes on said body or said door.

7. A medical waste disposal system comprising:
a medical waste container configured to receive medical waste for disposal;
a disposable supply of gloves configured for use in connection with the handling of the medical waste; and
a reusable glove box mounted with said medical waste container and housing said disposable supply of gloves, said glove box having a body at least partially defining an interior surface facing said disposable supply of gloves and an antimicrobial agent associated with said body in an amount effective to resist the growth of microbes on said body.

8. A medical waste disposal system comprising:
a disposable medical waste container configured to receive medical waste for disposal;
a reusable cart housing said disposable medical waste container, said reusable cart having a body at least partially defining an interior surface facing said disposable medical waste container and an antimicrobial agent associated with said body in an amount effective to resist the growth of microbes on said body.

9. A medical waste disposal system comprising:
a reusable medical waste container configured to receive medical waste for disposal, to be emptied of medical waste, and thereafter, to be reused to receive medical waste for disposal;
said reusable medical waste container having a body at least partially defining an interior surface further defining a medical waste receiving area and an antimicrobial agent associated with said body in an amount effective to resist the growth of microbes on said body.
